(19) United States
(12) Patent Application Publication
(10) Pub. No.: US 2026/0141979 A1
(43) Pub. Date: May 21, 2026

(54) Methods and Systems for Enhancing Protein Production

(71) Applicant: GENENET TECHNOLOGY (UK) LIMITED, London (GB)

(72) Inventors: Chi U. SEAK, London (GB); Chon In CHEONG, London (GB); Ming-Tsung Wu, London (GB); Chris Schultz, Redcar (GB); Liudmyla Bolotova, Redcar (GB); Suresh Ponnusamy, Redcar (GB); Cristina Parra Carmona, Redcar (GB)

(21) Appl. No.: 19/394,947

(22) Filed: Nov. 20, 2025

Related U.S. Application Data
(60) Provisional application No. 63/722,606, filed on Nov. 20, 2024.

(51) Int. Cl. G16B 25/10; C07K 14/47; C12N 5/071; C12N 9/10; G16B 5/20

(52) U.S. Cl. CPC G16B 25/10; C07K 14/4705; C12N 5/0686; C12N 9/1096; G16B 5/20; C12N 2510/00; C12Y 206/01052

(57) ABSTRACT

The present disclosure relates to protein production in mammalian cell lines using artificial neural network-like genetic circuits, and convolutional neural network predictions are described. A genetic circuit system including gene nodes selected based on roles in view of a target biological output is provided. The genetic circuit is applied to mammalian cells under bioproduction conditions, and the yield of the produced protein is measured.

### CROSS-REFERENCE TO RELATED APPLICATIONS

This application claims benefit of priority to U.S. Provisional Patent Application No. 63/722,606, filed on Nov. 20, 2024, the contents of which is hereby incorporated by reference in their entirety.

### TECHNICAL FIELD

The present disclosure relates to computational biotechnology and genetic engineering for recombinant protein expression in mammalian cell culture platforms.

### BACKGROUND

Biosynthetic processes increasingly rely on mammalian cell culture systems for the production of therapeutic proteins, such as monoclonal antibodies and recombinant proteins. Among these systems, Chinese Hamster Ovary (CHO) cells remain the dominant platform because they support human-like post-translational modifications and are highly adaptable to industrial bioprocessing conditions. Advances in genetic engineering have expanded our ability to modify cellular pathways and influence protein synthesis, metabolic flux, and cell growth. However, living cells possess highly complex gene-expression networks. Although various genetic engineering approaches are well established, identifying the right set of genes associated with the productivity of a target protein remains a time-consuming and labor-intensive process.

### BRIEF SUMMARY

In one aspect, the present disclosure provides an A genetic circuit system comprising: a data processing module configured to receive gene expression data for a plurality of genes from a plurality of biological samples; a feature selection module coupled to the data processing module and configured to identify a subset of key genes from the plurality of genes based on a target biological output; and a neural network model stored in a memory component and comprising a plurality of nodes arranged in a plurality of layers, wherein each node of the plurality of nodes corresponds to a gene of the subset of key genes and is associated with a gene-specific transfer function.

In one aspect, the present disclosure provides an engineered CHO cell, comprising an overexpressed sdhaf3 gene, an overexpressed psat1 gene, and an overexpressed fam83d gene.

### BRIEF DESCRIPTION OF THE SEVERAL VIEW OF THE DRAWING

FIG. 1 demonstrates an exemplary flowchart for the ANN-like genetic circuits design and cell engineering of the present disclosure.

FIG. 2 illustrates an exemplary flowchart for the ANN-like genetic circuits design and cell engineering of the present disclosure.

FIG. 3 illustrates the framework for the feature selection, model training, and the mimicANN simulation gene circuit design based on the time-series gene-metabolite feature dataset according to an example of the present disclosure.

FIG. 4A is a graphical representation showing the R² of each identified gene of an example of the present disclosure for predicting glucose levels.

FIG. 4B is a graphical representation showing the R² of each identified gene of an example of the present disclosure for predicting glutamine levels.

FIG. 4C is a graphical representation summarizing the overall R² gain from each additional gene feature across all metabolites, according to an example of the present disclosure, highlighting a saturation point where additional genes contribute marginal gains.

FIG. 4D is a graphical representation showing the R² of each identified gene of an example of the present disclosure for predicting glutamate levels.

FIG. 4E is a graphical representation showing the R² of each identified gene of an example of the present disclosure for predicting lactate levels.

FIG. 4F is a bar chart showing a Goodness-of-Fit plot according to an example of the present disclosure.

FIG. 4G is a graphical representation showing the R² of each identified gene of an example of the present disclosure for predicting IDH levels.

FIG. 4H is a graphical representation showing the R² of each identified gene of an example of the present disclosure for predicting ammonia levels.

FIG. 4I is a graphical representation showing a scatter plot of predicted versus actual values for one model (likely lactate or LDH), with a high R² of 0.9916 indicating excellent predictive performance.

FIG. 4J is a graphical representation showing how the model improved viability prediction accuracy over iterations.

FIG. 4K is a graphical representation showing a scatter plot of predicted versus true values over iterations.

FIG. 5A is a graphical representation showing normalized protein yield against different overexpression factors, 1.5, 2, 3, and 5 of the three key genes, sdhaf3 psat1, and/or fam83d at all states.

FIG. 5B is a graphical representation showing normalized protein yield against different overexpression factors, 1.5, 2, 3, and 5 of the three key genes, sdhaf3 psat1, and/or fam83d at a low yield state.

FIG. 5C is a graphical representation showing normalized protein yield against different overexpression factors, 1.5, 2, 3, and 5 of the three key genes, sdhaf3 psat1, and/or fam83d at a high yield state.

FIG. 5D is a graphical representation showing normalized viability against different overexpression factors, 1.5, 2, 3, and 5 of the three key genes, sdhaf3 psat1, and/or fam83d at all states.

FIG. 5E is a graphical representation showing normalized viability against different overexpression factors, 1.5, 2, 3, and 5 of the three key genes, sdhaf3 psat1, and/or fam83d at a low yield state.

FIG. 5F is a graphical representation showing normalized viability against different overexpression factors, 1.5, 2, 3, and 5 of the three key genes, sdhaf3 psat1, and/or fam83d at a high yield state.

FIG. 6A is a graphical representation showing response (yield) versus gene expression curves of sdhaf3 in individual overexpression.

FIG. 6B is a graphical representation showing response (yield) versus gene expression curves of psat1 in individual overexpression.

FIG. 6C is a graphical representation showing response (yield) versus gene expression curves of fam83d in individual overexpression.

FIG. 6D is a graphical representation showing response (yield) versus sdhaf3 gene expression curves when all three key genes (sdhaf3, psat1, fam83d) are simultaneously overexpressed.

FIG. 6E is a graphical representation showing response (yield) versus psat1 gene expression curves when all three key genes (sdhaf3, psat1, fam83d) are simultaneously overexpressed.

FIG. 6F is a graphical representation showing response (yield) versus fam83d gene expression curves when all three key genes (sdhaf3, psat1, fam83d) are simultaneously overexpressed.

FIG. 6G is a graphical representation showing response (viability) versus gene expression curves of sdhaf3 in individual overexpression.

FIG. 6H is a graphical representation showing response (viability) versus gene expression curves of psat1 in individual overexpression.

FIG. 6I is a graphical representation showing response (viability) versus gene expression curves of fam83d in individual overexpression.

FIG. 6J is a graphical representation showing response (viability) versus sdhaf3 gene expression curves when all three key genes (sdhaf3, psat1, fam83d) are simultaneously overexpressed.

FIG. 6K is a graphical representation showing response (viability) versus psat1 gene expression curves when all three key genes (sdhaf3, psat1, fam83d) are simultaneously overexpressed.

FIG. 6L is a graphical representation showing response (viability) versus fam83d gene expression curves when all three key genes (sdhaf3, psat1, fam83d) are simultaneously overexpressed.

FIG. 7 illustrates an exemplary flowchart for the CNN-based biomarkers selection and cell engineering of the present disclosure.

FIG. 8 is a bar chart showing that Trastuzumab protein yield (titre) in mg/L at 192 hours in an Ambr15 bioreactor for the control cell lines vs cell line overexpressing ANN plasmids (overexpressing the three genes: sdhaf3, psat1, fam83d).

FIG. 9 is a bar chart showing the amount of the metabolites (glutamine: 4 bars on the left, and lactate: 4 bars on the right) during Trastuzumab protein production of the control cell lines (control with empty vector transfection) vs cell line overexpressing ANN plasmids (overexpressing the three genes: sdhaf3, psat1, fam83d) at 192 hours in an Ambr15 bioreactor. Transient transfection with 1 ug/ml or 2.6 ug/ml of DNA plasmids shows consistent results for decreasing undesired metabolite (lactate) while increasing desired one (glutamine).

FIG. 10 is a graphical representation showing viability, VCC, and protein titer (protein yield, determined with IgG) throughout the production process (x-axis, in days) in an Ambr15 bioreactor with the control and ANN CHO cell line (overexpressing the three genes: sdhaf3, psat1, fam83d). The cells were tested at 37 degrees Celsius, 5% CO₂ and 125 rpm.

### DETAILED DESCRIPTION

Biosynthesis in pharmaceutical applications, particularly monoclonal antibody manufacturing, relies heavily on mammalian cell culture systems such as CHO cells. High protein yield, consistent product quality, and stable production characteristics remain a central challenge in industrial biosynthesis. Traditional approaches to genetic modification for yield improvement typically involve empirical testing of individual gene targets without systematic consideration of the complex regulatory networks and metabolic interactions within the cell. Such approaches lack precision and fail to capture the combinatorial effects of multiple genetic modifications acting in concert.

Existing methods for optimizing CHO cell productivity face several technical limitations. First, conventional genetic engineering strategies often focus on single-gene modifications without accounting for the interconnected nature of cellular metabolic and regulatory pathways. Second, the identification of optimal gene targets for modification typically relies on labor-intensive screening processes that do not leverage the predictive power of computational modeling. Third, the relationship between cellular metabolite profiles, gene expression patterns, and protein production outcomes remains poorly understood and underutilized in rational cell line engineering. Fourth, methods for systematically designing multi-gene modifications that mimic computational network architectures have not been applied to enhance bioproduction in mammalian cells.

Furthermore, the field lacks integrated approaches that combine mechanistic understanding of gene regulatory networks with data-driven predictive modeling to guide genetic modifications. The complexity introduced by combinatorial interactions between gene components under cellular context presents challenges for both the development and application of genetic circuit designs. Resource-sharing effects among genetic components and context-dependent behavior of regulatory parts further complicate the design process. Additionally, most reported methods for genetic circuit design have focused on diagnostic or therapeutic applications rather than fundamental enhancements to cellular production capacity in industrial bioprocessing settings.

In some examples, the present disclosure addresses these limitations by providing methods and systems that apply artificial neural network-like (ANN-like) genetic circuit principles to enhance protein production in mammalian cell lines. The approach leverages computational modeling to identify and implement multi-gene modifications that function as interconnected regulatory nodes, mimicking the architecture of artificial neural networks within the cellular environment.

In some examples, the present disclosure further provides convolutional neural network-based (CNN-based) predictive modeling methods that analyze correlations between cellular metabolites, biomarkers, and gene expression patterns to identify optimal genetic modification targets. The predictive modeling approach utilizes dimensionality reduction techniques applied to metabolite-gene correlation data to guide the selection of gene knockout or overexpression candidates that enhance production yield.

In some embodiments, the ANN-like genetic circuit method/system and the CNN-based predictive modeling method/system of the present disclosure can be performed or operated in tandem, as the CNN-based predictive modeling method/system can be implemented to identify key genes as an input for the ANN-like genetic circuit method/system.

In some examples, a method for enhancing protein yield in mammalian cell lines utilizes an artificial neural network-like genetic circuit comprising multiple gene nodes selected based on roles in metabolic regulation, cell cycle progression, and protein synthesis pathways. The genetic circuit design maps computational neural network principles to biological regulatory elements, where individual genes serve as nodes whose expression levels correspond to node outputs, regulatory interaction strengths between genes correspond to network weights, and basal promoter activities correspond to bias terms. In some embodiments, the method involves designing a genetic circuit that includes multiple gene nodes selected based on roles in metabolic and cell cycle regulation pathways relevant to protein synthesis, modulating expression of the selected genes to influence intracellular networks controlling protein synthesis, cell cycle progression, and metabolic efficiency, and applying the artificial neural network-like genetic circuit to mammalian cells under bioproduction conditions.

In some examples, the artificial neural network-like genetic circuit comprises specific gene nodes organized in a layered architecture. A first layer (e.g., an input layer) comprises input genes including creb3l1 (encoding a cAMP-responsive transcription factor involved in endoplasmic reticulum stress response), eid1 (encoding an EP300-interacting inhibitor of differentiation), ctsf (encoding cathepsin F, a lysosomal cysteine protease), ndufs5 (encoding a NADH: ubiquinone oxidoreductase subunit involved in mitochondrial electron transfer), tgm2 (encoding tissue transglutaminase involved in protein crosslinking and cellular signaling), adapt15 (a stress-inducible noncoding RNA), and tk1 (encoding thymidine kinase 1 involved in DNA precursor synthesis). A second layer (e.g., a first hidden layer) comprises sdhaf3 (encoding a regulator of gluconeogenesis and succinate metabolism) and psat1 (encoding phosphoserine aminotransferase 1 involved in serine biosynthesis). A third layer (e.g., a second hidden layer) comprises fam83d (encoding a positive regulator of cell cycle progression). The layered organization enables sequential signal propagation through the genetic circuit, where expression levels of genes in upstream layers influence expression of genes in downstream layers through regulatory interactions.

In some examples, the method involves the overexpression of specific gene nodes within the artificial neural network-like genetic circuit to enhance protein production. Overexpression of psat1 boosts serine biosynthesis pathways, contributing to increased availability of amino acid precursors for protein synthesis. Modulation of fam83d as a positive regulator of cell cycle progression enhances cell growth rates and protein yield by promoting cellular proliferation. Overexpression of sdhaf3 regulates gluconeogenesis and succinate metabolism, supporting metabolic efficiency and reducing cellular stress associated with high-density culture conditions. The combined overexpression of psat1, fam83d, and sdhaf3 in CHO cells results in a measurable increase in protein production compared to control cell lines, with observed yield improvements exceeding 15% for trastuzumab production in transient transfection experiments. Without wishing to be bound by any theories, even though the three genes' respective functions were known in the field, their respective roles in the complicated biosynthetic network of a CHO cell were not understood until the present disclosure identified their pivotal status in enhancing the CHO cell's production of recombinant proteins.

In some examples, the genetic circuit design incorporates gene-specific transfer functions that replace generic activation functions used in conventional artificial neural networks. Each regulatory interaction reflects experimentally measured gene-gene dynamics, capturing non-standard activation or repression curves specific to the biological context. The customization of transfer functions improves model fidelity and enables genetic circuit designs that predict biological behavior across diverse regulatory elements. The pre-activation affine transfer function of a given node combines weighted inputs from upstream regulators with a basal promoter activity term. The sigmoid activation function transforms the pre-activation value to produce the node output. For genetic component mapping, a promoter encodes the bias term and binding sites encode fan-in connections, where each regulator-site pair contributes a single regulatory weighted input. The transfer function and activation function are combined for each incoming edge and described in a single form that sums all incoming regulatory contributions, adds the promoter bias, and passes the result through the sigmoid function to generate the genetic node output.

In some examples, a method for predicting optimal genetic modifications for yield improvement in mammalian cell lines utilizes convolutional neural network technology to model gene-metabolite interactions. The method involves collecting metabolite and biomarker data from CHO cell cultures under various conditions and time points, calculating correlations between metabolite levels and gene expression levels using correlation metrics appropriate for the dataset characteristics (such as Spearman correlation for small sample sizes), and applying principal component analysis to the correlation data to reduce dimensionality and capture the most significant components of variation. The principal components derived from the metabolite-gene correlation data serve as input features for training a convolutional neural network model. The convolutional neural network is trained to identify and rank genetic modifications, including gene knockouts or overexpressions, that correlate with increased protein yield based on the embedded metabolite-gene relationship patterns captured in the principal components.

In some examples, the convolutional neural network architecture comprises multiple layers designed to process the principal component input data. A first one-dimensional convolutional layer with 64 filters and a kernel size of 4 extracts local patterns from the principal component features. An average pooling layer reduces dimensionality while retaining salient features. A second one-dimensional convolutional layer with 64 filters and a kernel size of 4 further processes the pooled features. A second average pooling layer performs additional dimensionality reduction. A flattening layer converts the multi-dimensional feature maps into a one-dimensional vector. A fully connected output layer with 128 parameters generates predictions. A final output layer with 2 parameters produces the target predictions for yield or viability. The network is trained with a dropout rate of 0.1, momentum of 0.85, and a learning rate of 0.0025 to prevent overfitting and ensure stable convergence.

In some examples, the convolutional neural network-based predictive model identifies specific gene targets for knockout modifications that enhance protein yield. The model predicts that knocking out the Tes gene (encoding a testis-derived protein) leads to reduced lactate production and improved metabolic efficiency. Transient CRISPR-based knockout of Tes in CHO cells results in a 20% decrease in lactate accumulation and a 10% increase in protein titer for trastuzumab production. The reduction in lactate levels is beneficial for bioproduction efficiency as lactate accumulation is associated with metabolic stress and reduced cell viability in high-density cultures. The model further predicts that knockout of the Uggt gene (encoding UDP-glucose: glycoprotein glucosyltransferase) optimizes protein processing within the endoplasmic reticulum and reduces the cellular burden associated with glycoprotein folding. Transient CRISPR-based knockout of Uggt in CHO cells results in a 10% increase in protein titer for trastuzumab production. The knockout modifications predicted by the convolutional neural network model have been validated in small-scale bioproduction settings, confirming improvements in yield under conditions representative of commercial production environments.

In some examples, the method involves applying both the artificial neural network-like genetic circuit overexpression strategy and the convolutional neural network-predicted gene knockout strategy in combination to achieve synergistic improvements in protein production. CHO cells are modified by the overexpression of psat1, fam83d, and sdhaf3 as part of an artificial neural network-like genetic circuit. Additionally, they are modified by CRISPR-based knockout of Tes to reduce lactate and improve metabolic efficiency, and by CRISPR-based knockout of Uggt to further increase protein yield through improved cellular processing of the therapeutic protein. The combined modifications result in cumulative yield improvements that exceed the effects of individual modifications applied separately.

In some examples, perturbation simulation studies are performed to validate and understand the causal effect of specific genes on predicted yield and viability. The perturbation studies involve establishing a baseline prediction for each sample in the dataset using the original gene expression profile with the trained model, and then systematically perturbing the expression levels of selected genes by applying scaling factors of 1.5, 2, 3, and 5 to simulate overexpression. For each perturbation, two scenarios are explored: scaling only the expression level of one modified gene while leaving all other genes unchanged, and scaling all modified genes by the scaling factor under test. The model prediction is recalculated for each perturbed sample, and the change in predicted yield or viability is recorded. Gene expression versus predicted yield curves are plotted for each perturbed gene, and the linearity, saturation point, monotonicity, biological plausibility, slope, and curvature of the response curves are analyzed. The perturbation studies reveal that sdhaf3 overexpression shows a consistently positive relationship with protein yield across all scaling factors, with yield rising more steeply at higher scaling levels, indicating that sdhaf3 is a strong candidate for upregulation. The psat1 gene exhibits only a modest positive effect on yield at higher scaling factors, with curves remaining relatively flat for lower scale values, suggesting that psat1 has a limited and possibly saturating influence on yield. The fam83d gene demonstrates a negative correlation with protein yield in individual overexpression scenarios, but the scaling has an overall effect of increasing yield across all target genes within the final two layers when considered in the context of the full genetic circuit, suggesting complex regulatory interactions and bias effects.

In some examples, the perturbation simulation results inform the design of genetic modifications by revealing synergistic effects among multiple gene modifications. Although individual overexpression of genes results in amplified gradients and dynamic range, the absolute values of predicted yield may remain below baseline in some cases, indicating down-regulation effects. However, when multiple genes are overexpressed together, such as the triple overexpression of sdhaf3, psat1, and fam83d, the combined effect results in a substantially higher yield than any individual overexpression. The synergistic effect is attributed to the cascaded boosting of bias terms across the multiple layers of the genetic circuit, which increases the dynamic range of protein yield. Without wishing to be bound by theories, in some embodiments, it was observed from the perturbation studies that psat1 serves as a viability balancing element. Those embodiments reveal that psat1 is the only gene among the tested targets that showed an increasing effect on viability over scaling factors, suggesting that psat1 overexpression might help maintain cell viability while other modifications focus on enhancing yield.

In some examples, a biosynthesis or bioproduction system for therapeutic proteins in mammalian cell lines integrates the artificial neural network-like genetic circuit and the convolutional neural network-based predictive model into a unified platform. The system comprises an artificial neural network-like genetic circuit designed to modulate expression of key genes related to cellular metabolism and protein synthesis, a convolutional neural network-based predictive model that uses principal component analysis on metabolite and biomarker data to identify further gene modifications, a process for implementing modifications identified by the convolutional neural network model and artificial neural network-like circuit in mammalian cell cultures to enhance production of therapeutic proteins, and a system for real-time monitoring of cellular metabolites and biomarkers to dynamically adjust cell culture conditions, optimizing yield and metabolic stability.

In some examples, the method involves dynamically optimizing protein yield in bioproduction using artificial neural network and convolutional neural network technologies. The dynamic optimization involves periodically adjusting gene expression within the artificial neural network-like circuit based on real-time monitoring of biomarkers such as lactate, serine, and glucose levels in the culture medium. The convolutional neural network model is used to reassess and update gene modification recommendations based on evolving metabolite and yield data collected during the production run, ensuring continued high-yield production of the target protein. The dynamic optimization approach allows the bioproduction system to adapt to changing cellular states and culture conditions, maintaining optimal productivity throughout the production cycle.

In some examples, the methods and systems are applied to produce trastuzumab, a monoclonal antibody used in cancer therapy, in CHO cells. The cell line is modified by both artificial neural network-like genetic circuit overexpression and convolutional neural network-predicted gene knockouts to enhance production. Experimental validation demonstrates that the combined modifications result in over 15% increase in trastuzumab titer in transient transfection experiments, with additional improvements in metabolic efficiency as evidenced by reduced lactate accumulation. The methods are applicable to other therapeutic proteins and monoclonal antibodies produced in CHO cells or other mammalian cell lines.

In some examples, the biomarker search algorithm utilizes machine learning techniques, including XGBoost and SHAP (Shapley Additive explanations) analysis, to identify key gene features that contribute to predicting metabolite levels and production outcomes. The algorithm performs incremental feature selection, evaluating the R² performance of individual genes in predicting specific metabolites, including glucose, glutamine, glutamate, lactate, lactate dehydrogenase (LDH), and ammonia. The analysis reveals that a subset of genes holds strong predictive power for multiple metabolites, enabling efficient modeling with a limited feature set. The diminishing returns of R² as additional gene features are included imply that only a few key gene features capture most of the signal in multivariate models. The identified key genes, including creb3l1, ndufs5, and psat1, contribute more significantly than others to metabolic prediction, and these genes are prioritized for inclusion in the artificial neural network-like genetic circuit design.

In some examples, the methods achieve high predictive performance as demonstrated by goodness-of-fit metrics (R²). For metabolite prediction models, R² values exceeding 0.99 are achieved for lactate and LDH predictions, indicating excellent predictive performance. For viability prediction, the model achieves an R² of 0.71 after iterative training, with strong agreement between predicted and true viability values. For yield prediction, the model achieves an R² of 0.71, demonstrating robust predictive capability for protein production outcomes. The high predictive performance validates the utility of the artificial neural network-like genetic circuit design and convolutional neural network-based modeling approaches for guiding genetic modifications in bioproduction systems.

In some examples, the disclosed methods provide advantages over conventional approaches to cell line engineering for bioproduction. The artificial neural network-like genetic circuit design enables systematic multi-gene modifications that account for complex regulatory interactions and metabolic networks within the cell, rather than relying on empirical single-gene modifications. The convolutional neural network-based predictive modeling leverages metabolite-gene correlation data to identify optimal modification targets, reducing the need for labor-intensive screening and enabling data-driven rational design. The integration of mechanistic understanding through the genetic circuit design with data-driven prediction through the convolutional neural network model provides a comprehensive platform for cell line optimization. The methods have been validated in experimental settings with commercially relevant therapeutic proteins, demonstrating practical applicability and scalability to industrial bioproduction processes.

### Example 1: ANN-Like Genetic Circuit Design and Implementation

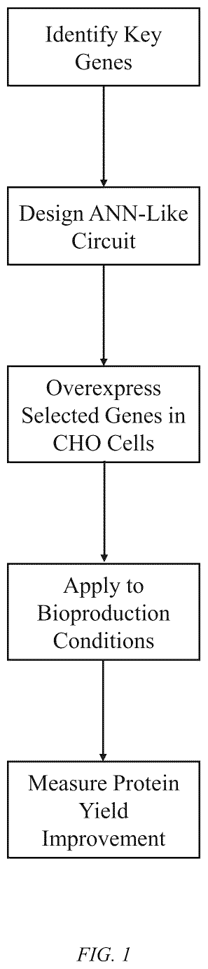

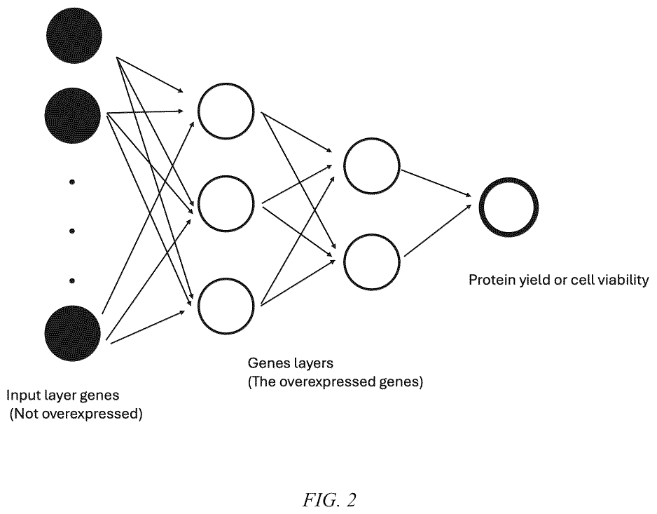

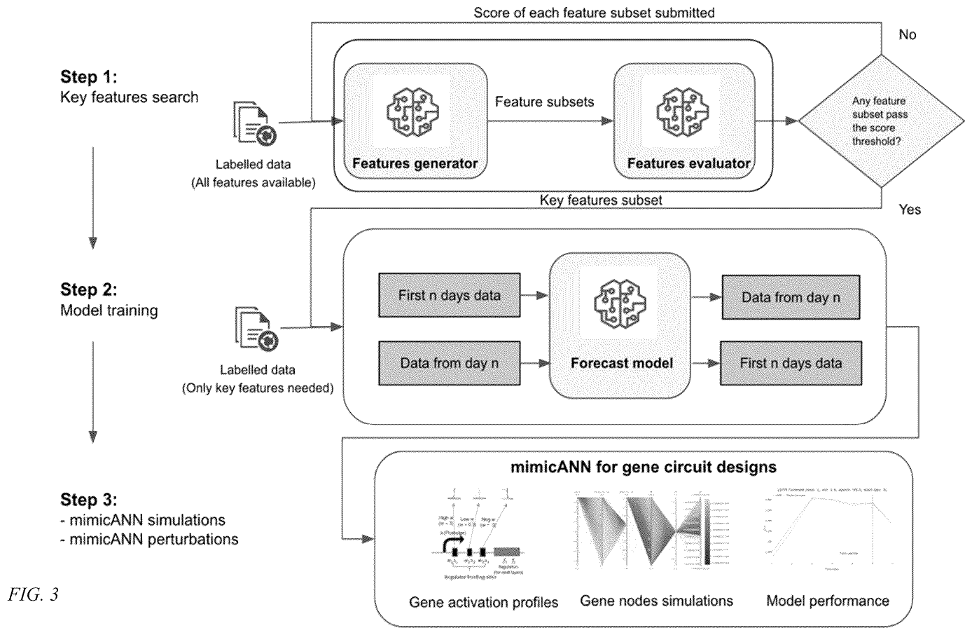

The ANN-inspired approach (FIG. 1, FIG. 2, and FIG. 3) applies genetic engineering to create synthetic circuits in CHO cells, which mimic neural networks.

Dataset description. The data collection procedure aimed at investigating gene expression changes over time or across conditions, to model and explain variations in yield or viability of CHO cells. The dataset contains gene expression for 44 experimental samples, each with a unique condition identifier and time point. There were two target variable columns, namely viability (coded: via) and yield (coded: octet_protein_titration), and the gene columns consisting of the expression levels for over 1291 genes, represented by both gene names and locus identifiers, without missing data.

Genetic circuit design algorithm. The genetic circuit formula maps ANN computation to biological regulatory elements, from ANN node to gene-regulatory node. The ANN nodes were represented by genes whose expression levels (protein/RNA concentrations) correspond to the node's output. The weights in the ANN were implemented as the strengths of regulatory interactions between genes—for example, the affinity of a transcription factor for its binding site. The bias terms corresponded to basal promoter activity (gene expression in the absence of regulators).

By replacing the generic activation functions with gene-specific transfer functions, this allows each regulatory interaction to reflect experimentally measured gene-gene dynamics. This customization captures non-standard activation or repression curves, improving model fidelity and enabling genetic circuit designs that predict real biological behavior across diverse regulatory elements in a realistic context.

The pre-activation affine transfer function of a given node j:

$${x}_{t}^{( j )} = {\sum }_{i} {w}_{ji} {x}_{i} + {b}_{j}$$

The sigmoid activation function with the output node of j is defined as

$${y}_{j} = σ ( {x}_{t}^{( j )} ) : f ( x ) = σ ( x ) = \frac{1}{1 + {e}^{- x}}$$

Whereas, for the mapping of genetic components:

$${y}_{i} = σ ( {x}_{t}^{( i )} ) = \frac{1}{1 + {e}^{- {x}_{t}^{( i )}}} .$$

With a promoter encodes the bias bⱼ and the binding sites encode the fan-in; and each regulator-site pair contributes a single regulatory weighted input. Because a single regulator-site implements one regulation, here the transfer function and activation function are combined for each incoming edge, therefore described in a single form:

$${Regulation}_{ji} = {w}_{ji} {x}_{i} = {w}_{ji} σ ( {x}_{t}^{( i )} ) = \frac{{w}_{ji}}{1 + {e}^{- {x}_{t}^{( i )}}}$$

Summing all incoming regulatory contributions and adding the promoter bias, then passing through the sigmoid gives the genetic node output:

$${y}_{j} = σ ( {b}_{j} + {\sum }_{i} {w}_{ji} σ ( {x}_{t}^{( i )} )$$

With the recursion for the previous layer. This reduces the familiar ANN form implemented by genetic parts:

$${y}_{j} = σ ( {b}_{j} + {\sum }_{i} {w}_{ji} {y}_{i} )$$

Where yⱼ is the concentration/activity produced by upstream regulators.

The genetic circuit design pipeline. ANN-like genetic circuits were developed to enhance yield. The full genes, hereby the “nodes” in the circuit, of the simulated mimicANN were as described in the following Table 1.

TABLE 1

| Gene Name | Exemplary Gene Function |
|---|---|
| creb311 | Encodes a cAMP-responsive transcription factor (OASIS) embedded in |
| | the ER membrane. Under ER stress, it's cleaved, and the released |
| | N-terminal domain translocates to the nucleus, activating genes via box- |
| | B elements. |
| eid1 | EP300-interacting inhibitor of differentiation 1; binds EP300/CBP and |
| | RB1, represses MYOD1 transactivation, and may link cell cycle exit to |
| | differentiation by inhibiting histone acetyltransferase activity. |
| ctsf | Cathepsin F, a lysosomal cysteine protease essential for protein |
| | degradation, endosomal/lysosomal trafficking, antigen processing, and |
| | implicated in neuronal ceroid-lipofuscinosis when mutated. |
| ndufs5 | NADH: ubiquinone oxidoreductase subunit S5, an accessory iron-sulfur |
| | protein in mitochondrial complex I, facilitating electron transfer from |
| | NADH to ubiquinone. |
| tgm2 | Tissue transglutaminase (TG2), a calcium-dependent enzyme that |
| | crosslinks proteins via γ-glutamyl-lysine bonds, with additional GTPase, |
| | deamidase, isopeptidase, and signaling roles; involved in extracellular |
| | matrix stabilization, fibrosis, and epigenetic histone modifications |
| adapt15 | A stress-inducible noncoding RNA transcript upregulated by hydrogen |
| | peroxide; associates with polysomes and likely acts at the translational |
| | level to protect cells from oxidative damage. |
| tk1 | Thymidine kinase 1, a cytosolic enzyme peaking in S-phase that |
| | phosphorylates thymidine, regulating the DNA precursor pool; activity |
| | is low in resting cells and elevated in proliferating and cancer cells. |
| sdhaf3 | A regulator of gluconeogenesis and succinate metabolism |
| psat1 | A gene that is involved in serine biosynthesis. |
| fam83d | A positive regulator of cell cycle progression. |

The value of the weights between the network layers, each with a gene-gene specific activation, are listed in Table 2 below. The network composition of the gene nodes was:
Layer 1 (input layer): creb3l1, eid1, ctsf, ndufs5, tgm2, adapt15, tk1, and
Layer 2: sdhaf3, psat1; Layer 3: fam83d.

TABLE 2

| | Layer | | | |
|---|---|---|---|---|
| | Layer 0-1 | Layer 1-2 | Layer 2-3 | Layer 3-4 |
| Gene | (Input layer) | (Hidden layer) | (Hidden layer) | (Output layer) |
| Gene node 1 | −0.05453148 | 0.3374743 | −0.26206 | −0.5754732 |
| Gene node 2 | 0.43099052 | 0.22920588 | 0.70296586 | |
| Gene node 3 | −0.50655013 | −0.22527301 | | |
| Gene node 4 | −0.24100842 | −0.43830562 | | |
| Gene node 5 | −0.27266786 | −0.30757898 | | |

The application of mimicANN. It implemented an explainable AI pipeline using a mimic neural network model that mimics a black-box predictor trained on gene expression data (specifically focusing on overexpressed genes). The goal was to identify genes associated with yield and viability using interpretable methods with topological optimization and perturbation-based simulation studies.

### Algorithm

1. Data Preparation: With inputs of (i) Gene expression matrix (features), (ii) yield, viability (or both) data (target), and (iii, optional) DEGs list for gene filtering.
2. Preprocessing: Log transformation, normalization, and filtering of genes based on variance or overexpression criteria.
3. Model Training: A fully connected neural network (standard MLP) was trained to predict yield from expression data. This is the “teacher model”.
4. Explainable Model Training: After the teacher was trained, its predictions were used as targets to train an interpretable model, which tries to mimic the teacher's behavior. Two main interpretable schemes were considered for the customization of gene nodes activation functions: (i) Polynomial regressors, (ii) Shallow Decision Tree, (iii) Sparse Linear Models (e.g., Lasso Regression).
5. Model Evaluation: The R² of the mimicANN model inference (compared to the ground truth via the goodness-of-fit plot) for each objective target (hereby yield and viability), and the value-propagated activation curves of the gene nodes were presented for both performance and domain-specific explainability evaluations.
6. Gene selection for downstream: The most important genes were selected and exported for downstream use. Downstream applications include plasmid designs.

The perturbation studies of mimicANN found gene nodes. The goal of the perturbation studies was to validate and understand the causal effect of specific genes on the predicted yield and viability as determined by the model. With the following questions being answered:
Which genes have the strongest causal influence on yield predictions?
Whether the model's reliance on a gene is robust, or possibly spurious?
How does changing a single gene expression level alter the output?

Firstly, a baseline prediction was established. For each sample in the dataset, predict yield using the original gene expression profile with the trained black-box model. For the perturbation process, for each gene, g, in the modified key gene list, loop through a range of synthetic over-expression values, specifically with scaling factors of 1.5, 2, 3, and 5. For each value, two scenarios had been explored: (i) scale only the expression level of one of the modified genes (leave all other genes unchanged) and (ii) scale all of the modified genes by the scaling factor under test. Then, recalculate the model prediction for each perturbed sample and record how the model prediction changes. The gene expression vs predicted yield for each perturbed gene was plotted. The linearity, saturation point, monotonicity, biologically plausibility, slope, and curvature of the response curve are subject to analysis.

Bioassay. Plasmids containing overexpression constructs for Psat1, Fam83d, and Sdhaf3 were designed and validated for expression in CHO cells. After 168-192 hours, the protein yield was measured in the culture medium and compared to that of non-transfected control CHO cells.

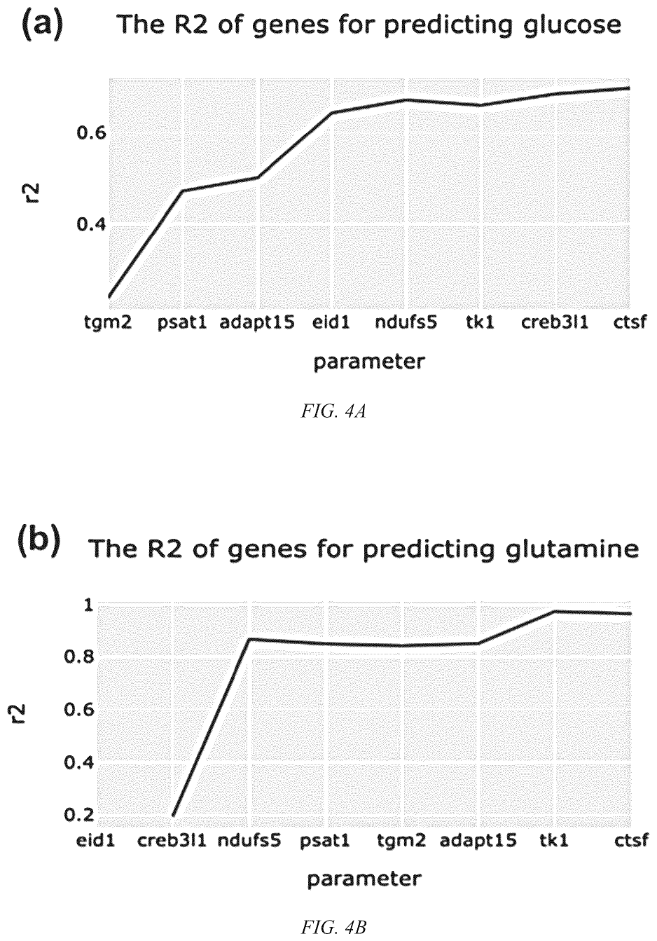

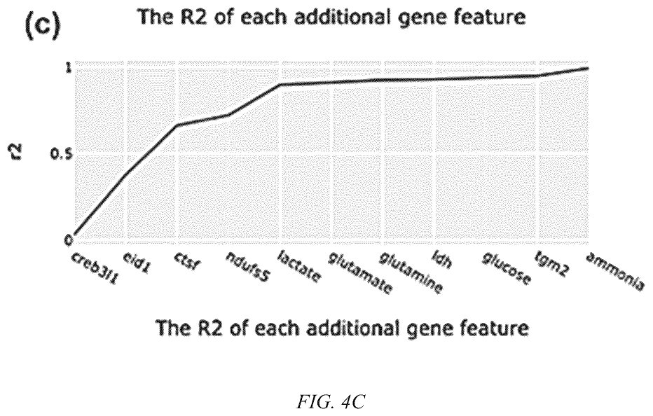

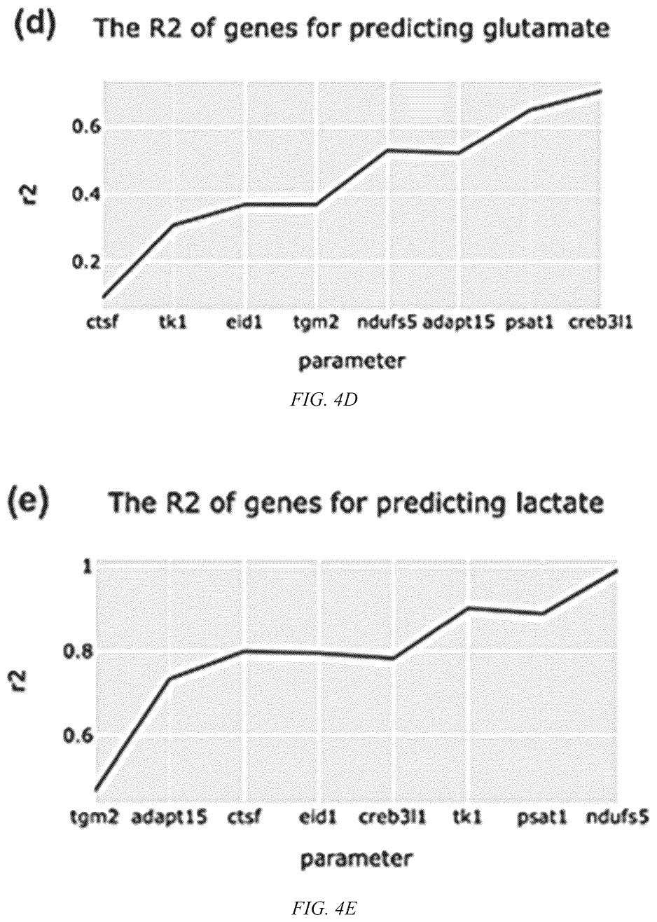

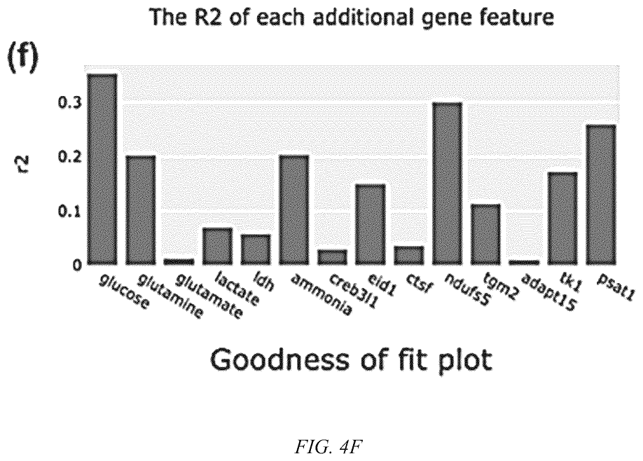

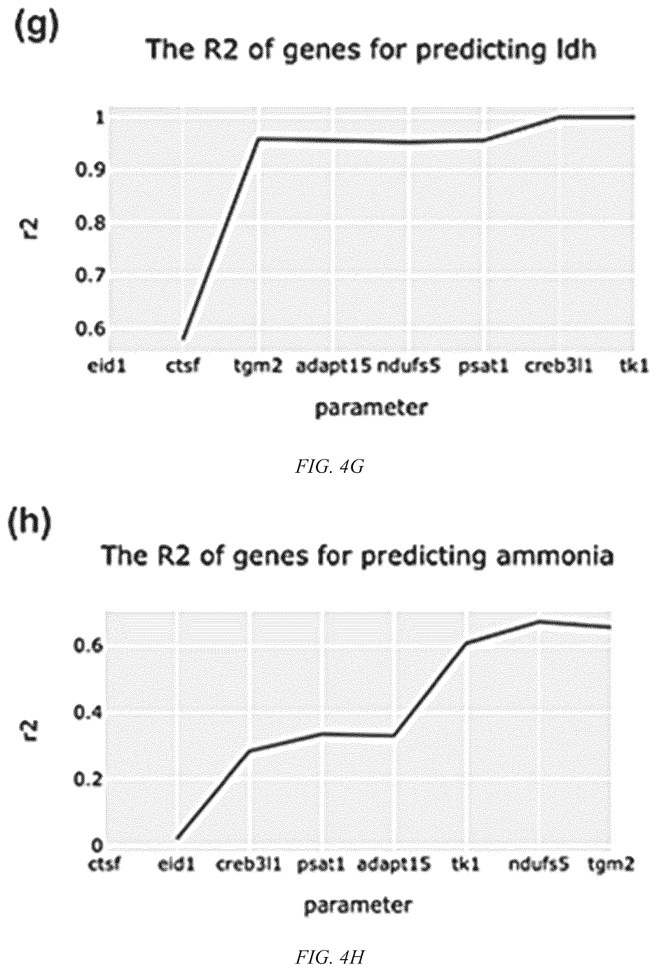

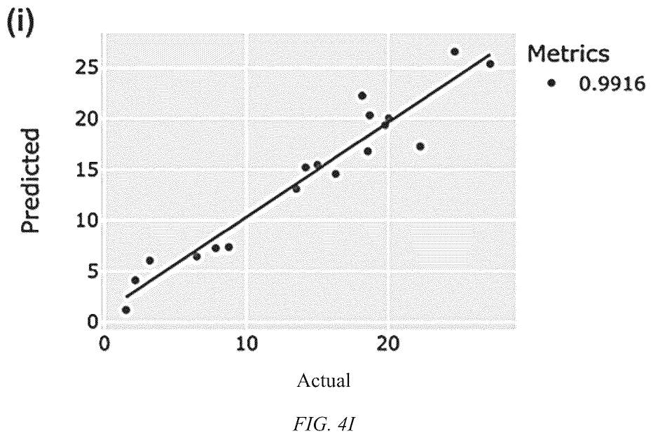

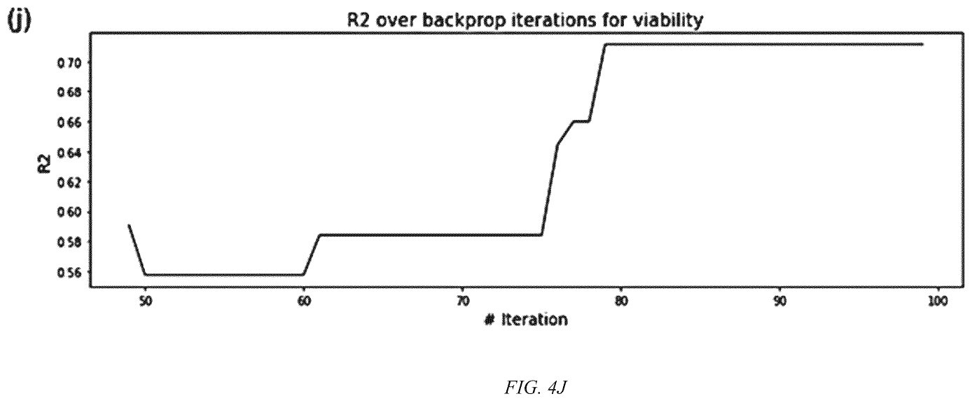

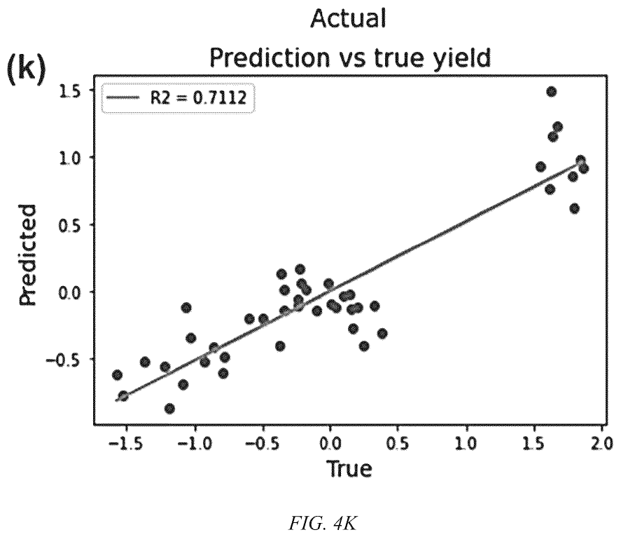

Result. FIG. 4 shows the incremental R² performance of individual genes in predicting specific metabolites, including glucose, glutamine, glutamate, lactate, LDH, and ammonia. Each plot illustrates how predictive power increases as additional genes are included, with specific genes, such as creb3l1, ndufs5, and psat1, contributing more significantly than others. FIG. 4C. summarizes the overall R² gain from each additional gene feature across all metabolites, highlighting a saturation point where additional genes contribute marginal gains. FIG. 4F. is a bar chart comparing the goodness-of-fit (R²) of each gene feature across all metabolite models, emphasizing key contributors. Finally, FIG. 4I. shows a scatter plot of predicted versus actual values for one model (likely lactate or LDH), with a high R² of 0.9916 indicating excellent predictive performance. Overall, the figure suggests that a subset of genes holds strong predictive power for multiple metabolites, enabling efficient modelling with a limited feature set.

These findings aligned with previous studies highlighting the utility of transcriptomic features for metabolic prediction. For example, the PSAT1 gene has been reported to be related to the amino acid metabolism and biosynthesis; furthermore, it has strong and positive correlations with the shear stress. Through comparative transcriptome analysis, it has been reported that the gene NDUFS5 is associated with increased recombinant protein productivity in mammalian cells. The high R² values observed, particularly for lactate and LDH, suggest a gene-metabolite relationship that may support targeted biomarker discovery and metabolic engineering.

Overall, the diminishing returns of R² in FIG. 4C. implied a pattern in multivariate models where only a few key gene features can capture most of the signal. FIG. 4J and FIG. 4K showed how the model improved viability prediction accuracy over iterations, reaching an R² of 0.71, with strong agreement between predicted and true yield values in the final performance plot.

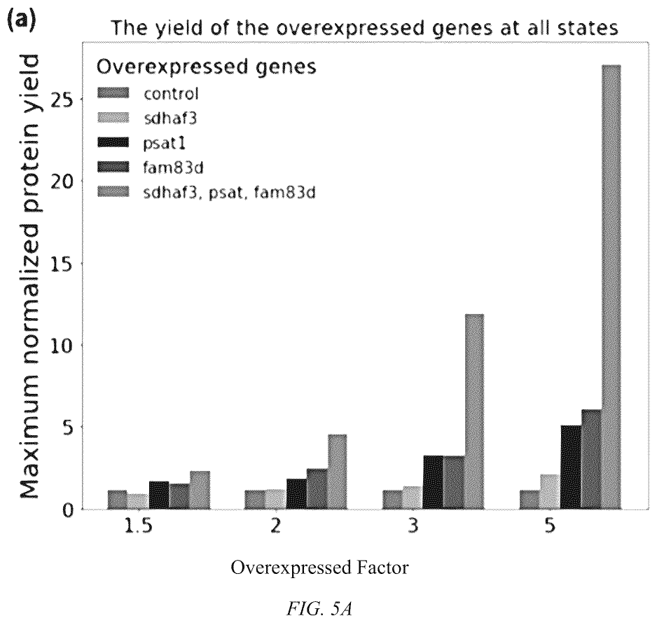

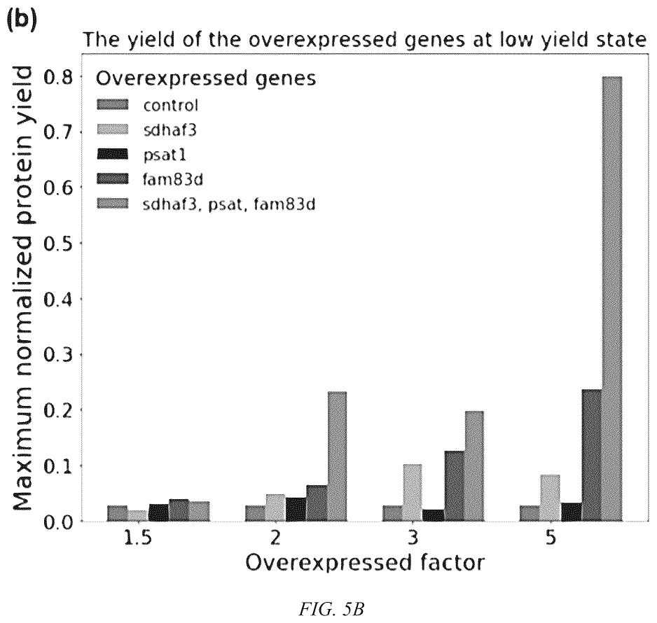

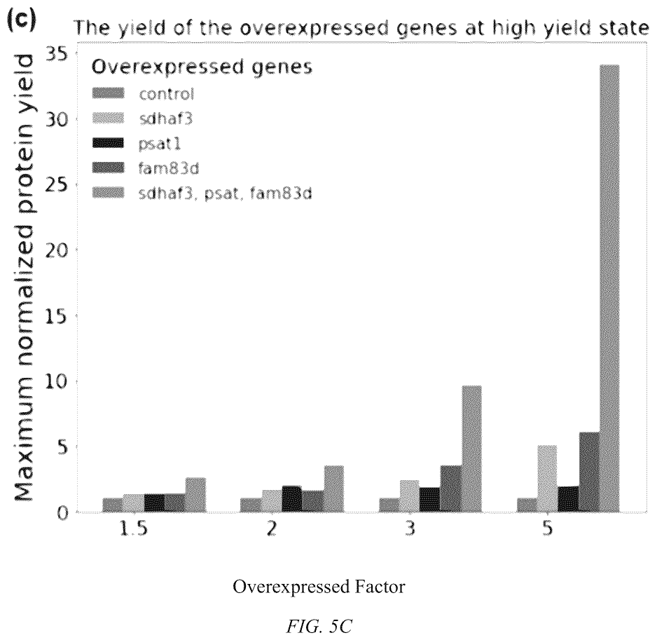

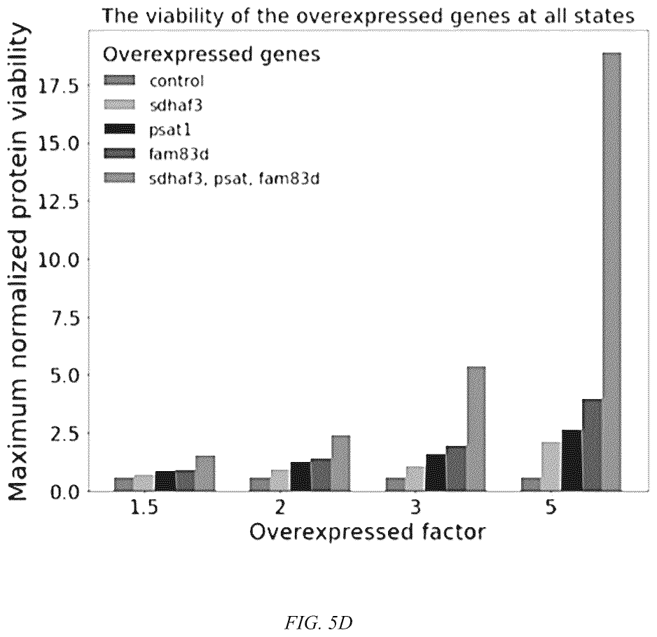

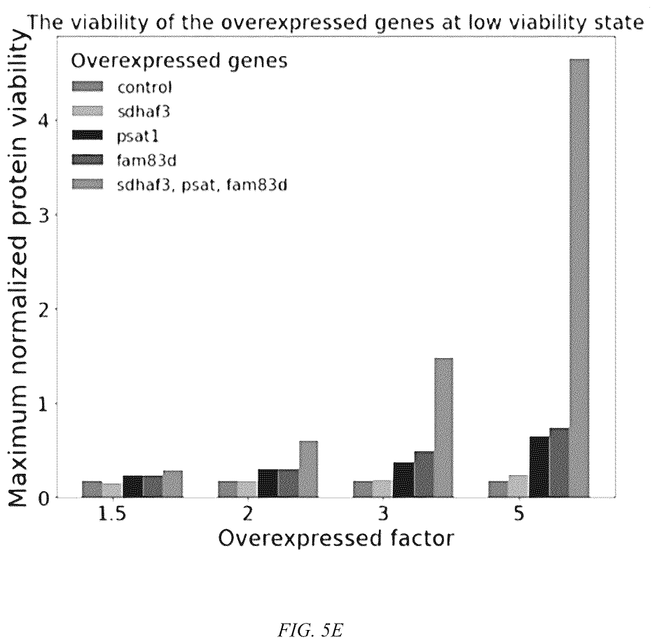

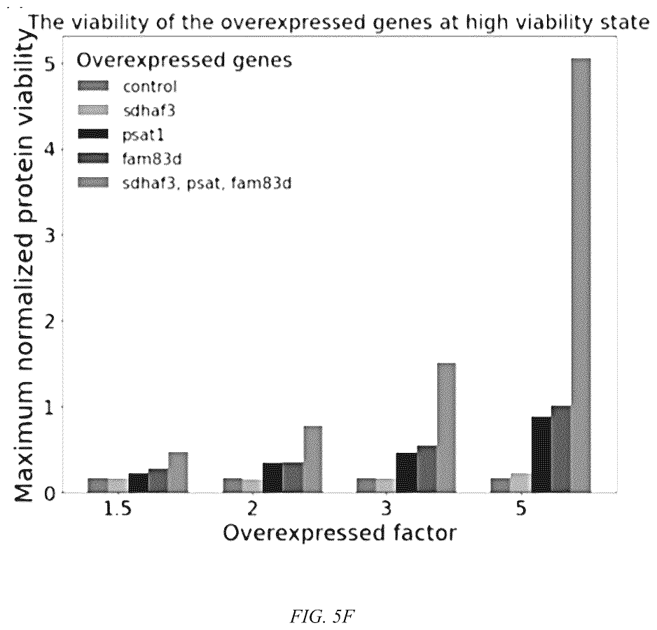

In FIG. 5A-FIG. 5F, while PSAT1 supports nucleotide and amino acid synthesis, its overexpression alone may not significantly enhance metabolic flux unless accompanied by upregulation of upstream or downstream enzymes. Additionally, PSAT1 may divert glycolytic intermediates away from central energy metabolism, creating a resource trade-off that limits productivity and growth. The markedly higher performance observed in the triple overexpression condition (sdhaf3.1, psat1, fam83d) suggests that PSAT1's effects are synergistic when mitochondrial support (via SDHAF3.1) and cell cycle regulation (via FAM83D) are also enhanced. Thus, PSAT1 contributes to metabolic output, but only achieves substantial gains in combination with complementary functions.

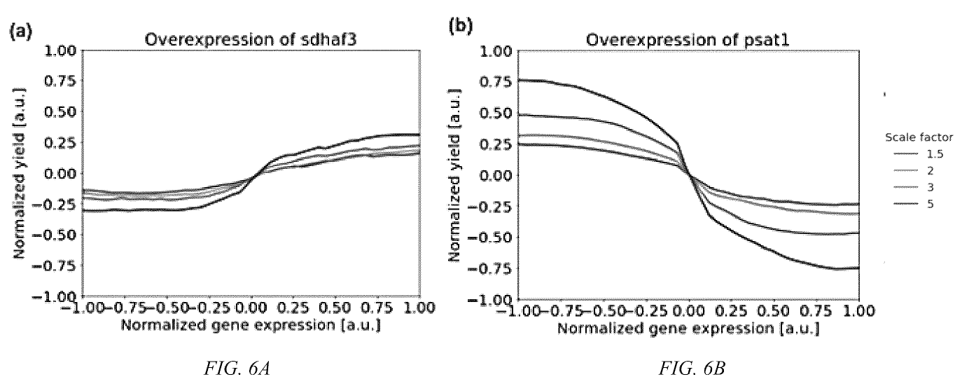

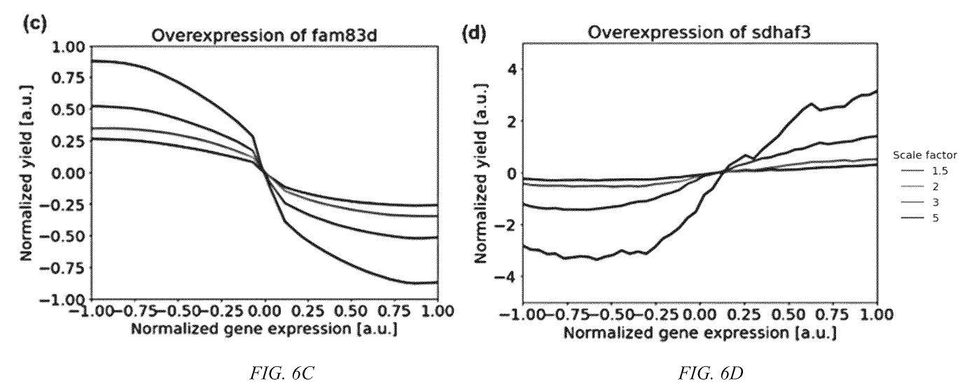

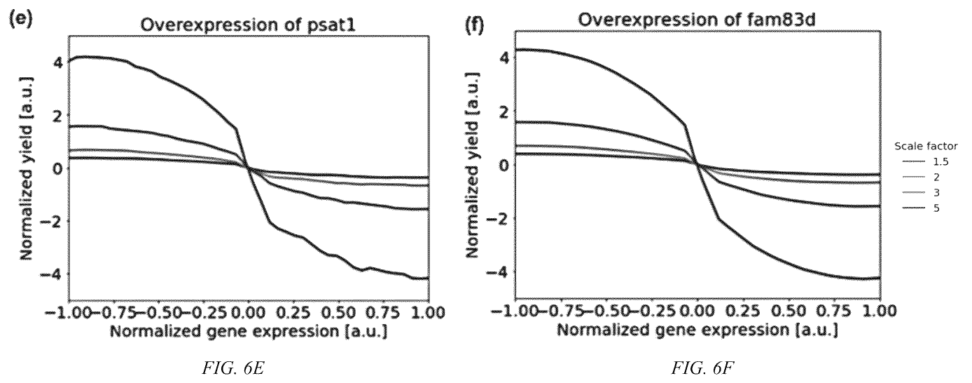

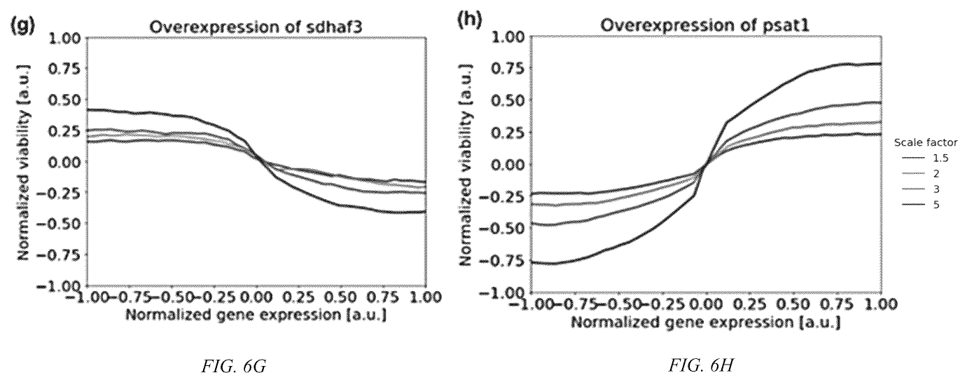

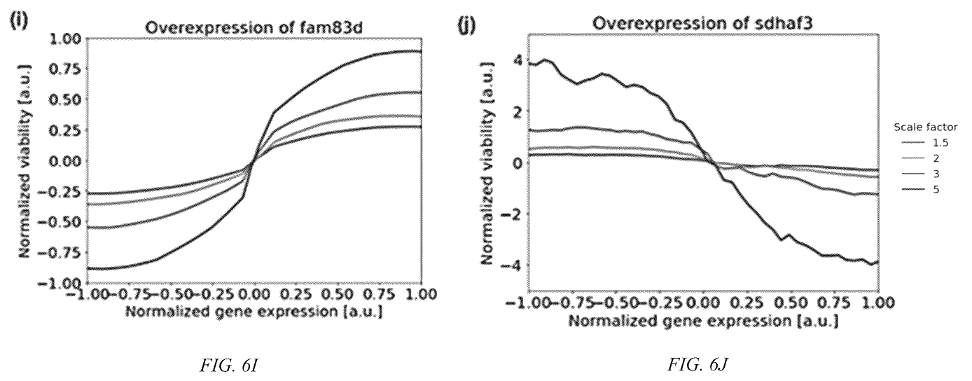

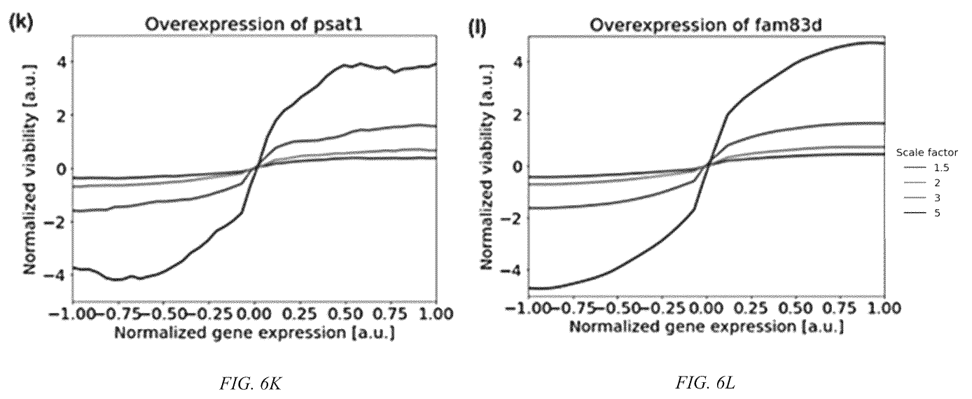

As shown in FIG. 6A-FIG. 6C, although the individual overexpression genes resulted in amplified gradients and dynamic range, the absolute values are always below 1 (one) and thus down-regulation. In FIG. 6D-FIG. 6F, while SDHAF3 and FAM83d overexpression boosted the yield, PSAT1 overexpression results in declined. This highlights gene-specific, nonlinear relationships between expression and yield, with greater effect magnitude at higher perturbation scales. The synergetic effect is critical to be considered for the perturbation of the three key genes. The normalized viability in FIG. 6J-FIG. 6L highlights the importance of PSAT1 for the viability balancing element, as PSAT1 is the only gene with an increasing effect for viability over scales (Table 3).

TABLE 3

| | Impact on | Effect of | Potential |
|---|---|---|---|
| Gene | Yield | Scale Factor | Strategy |
| sdhaf3 | Positive | Increased | Overexpression |
| | | dynamic | and keep the genes |
| | | range | of its input |
| | | | layer high |
| psat1 | Positive | Increased | Overexpression |
| | | dynamic | while keeping low |
| | | range | input of genes in |
| | | | the previous layer |
| fam83d | Positive | Positive | Overexpression |
| | | offset effect | for scaling bias |

The provided plots analyzed the effect of gene overexpression on protein yield under varying scaling factors. The top row features three-line plots that display how normalized protein yield changes with normalized gene expression levels for three genes: sdhaf3, psat1, and fam83d. Each colored line represents a different scaling factor (1, 1.5, 2, 3, and 5), corresponding to different degrees of simulated gene overexpression. These plots aim to capture the sensitivity of protein yield to changes in expression levels of each gene under increasingly exaggerated biological conditions.

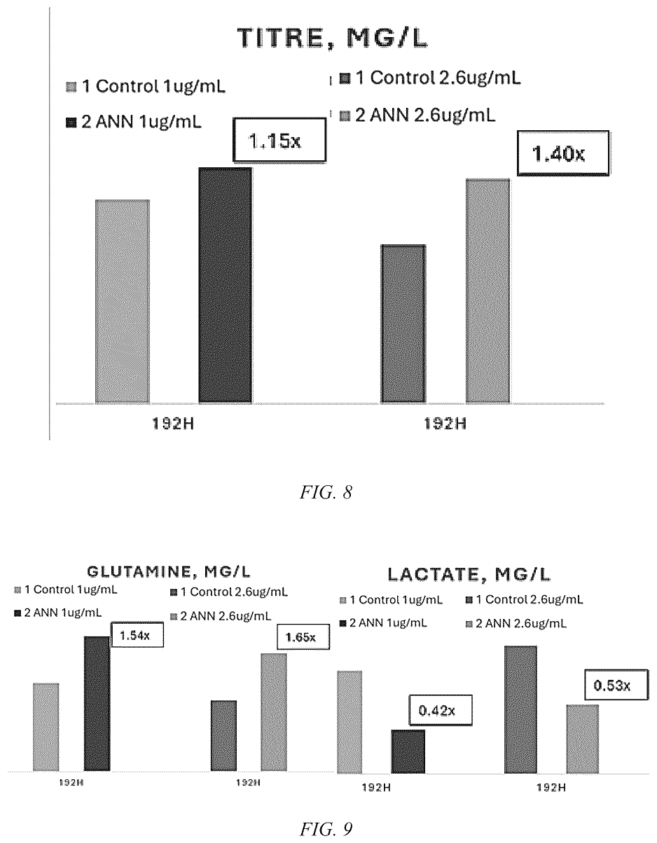

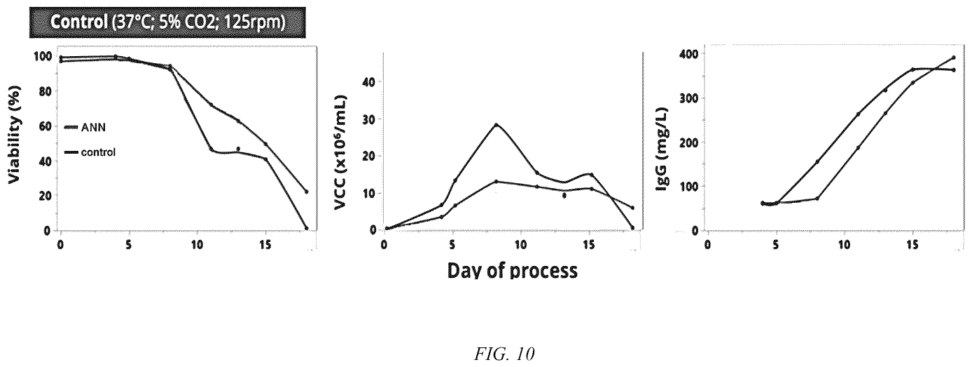

The experimental group showed a statistically significant increase in yield, reaching over 15% improvement for trastuzumab titer (FIG. 8). It was also noticed that the Trastuzumab titer increase aligned with decreasing undesired metabolite (lactate) while increasing desired one (glutamine) (FIG. 9). Furthermore, the viability, VCC, and protein titre (protein yield, determined with igG) throughout the production process were shown in FIG. 10.

The sdhaf3 gene shows a consistently positive relationship with protein yield across all scaling factors. As gene expression increases, the normalized yield rises more steeply with higher scaling, especially at a scale factor of 5. This indicates that sdhaf3 is a strong candidate for upregulation, as its overexpression robustly enhances yield. In contrast, the psat1 gene exhibits only a modest positive effect on yield, and this effect is only observed at higher scaling factors. The curves remain relatively flat for lower scale values, suggesting that psat1 has a limited and possibly saturating influence on yield. Lastly, fam83d demonstrates a negative correlation with protein yield. However, the scaling has an overall effect of increasing the yield across all the target genes within the final two layers, as suggested by the mimicANN.

### Example 2: CNN-Predicted Knockout Strategy for Yield Improvement

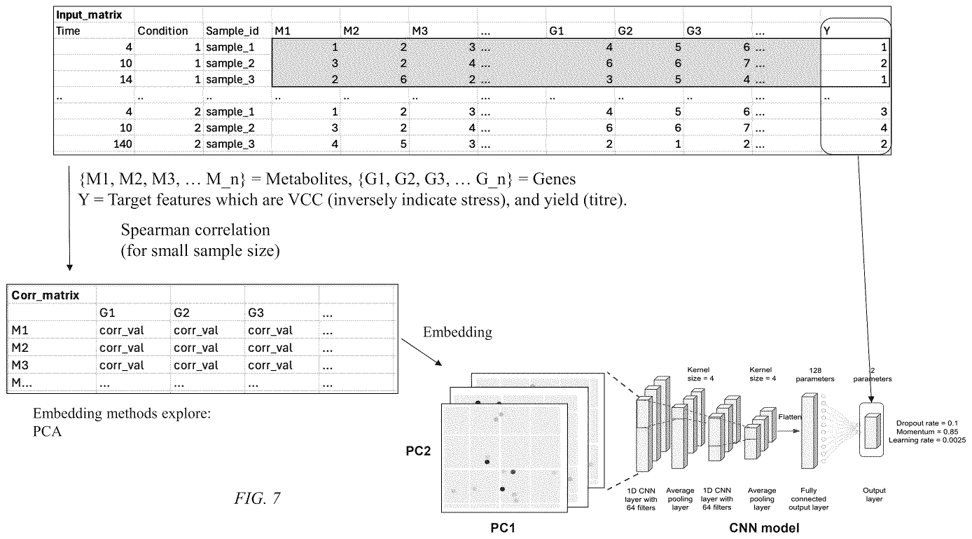

A CNN model was developed to predict gene modifications that enhance protein yield by using PCA of metabolite and biomarker correlations (FIG. 7). Tes and Uggt were identified as candidate genes where knockouts could favorably impact production efficiency.

Model Development. Using a dataset of metabolite-gene correlations obtained from CHO cell cultures, Spearman correlations were calculated, and Principal Component Analysis (PCA) was applied to reduce dimensionality, capturing the most significant components. These principal components were used as input features for CNN training, targeting yield improvement predictions.

Validation of Gene Targets in Small-Scale Bioproduction. CHO cells were transiently modified via CRISPR plasmids to knock out Tes and Uggt. After a 48-hour culture period, titers were measured, revealing a 10% increase in trastuzumab yield in both Tes and Uggt knockout cells. Additionally, the Tes knockout resulted in a 20% decrease in lactate accumulation, thereby reducing metabolic stress and improving overall bioproduction efficiency.

Conclusion. This CNN-guided approach demonstrates the effectiveness of predictive modeling for identifying genetic modifications that enhance yield in CHO cells. The method is applicable across a range of therapeutic proteins and can be scaled for high-efficiency production.

What is claimed is:

1. A genetic circuit system comprising:
a data processing module configured to receive gene expression data for a plurality of genes from a plurality of biological samples;
a feature selection module coupled to the data processing module and configured to identify a subset of key genes from the plurality of genes based on a target biological output; and
a neural network model stored in a memory component and comprising a plurality of nodes arranged in a plurality of layers, wherein each node of the plurality of nodes corresponds to a gene of the subset of key genes and is associated with a gene-specific transfer function.

2. The genetic circuit system of claim 1, further comprising a mimic model generation module coupled to the neural network model and configured to generate an interpretable surrogate model that approximates predictions of the neural network model, wherein the interpretable surrogate model comprises a plurality of gene regulatory nodes arranged in a layered architecture.

3. The genetic circuit system of claim 2, further comprising a perturbation simulation module coupled to the mimic model generation module and configured to simulate expression level perturbations for one or more genes of the subset of key genes by applying a plurality of scaling factors to baseline gene expression values.

4. The genetic circuit system of claim 3, further comprising an output module coupled to the perturbation simulation module and configured to generate predicted values for the target biological output based on the simulated expression level perturbations.

5. The genetic circuit system of claim 1, wherein the target biological output comprises protein yield, cell viability, or a combination thereof.

6. The genetic circuit system of claim 1, wherein the gene-specific transfer function for each node comprises gene expression data that reflects experimentally measured gene-gene dynamics.

7. The genetic circuit system of claim 6, wherein the gene-specific transfer function for each node further comprises a regulatory interaction with another node of a plurality of nodes.

8. The genetic circuit system of claim 2, wherein the interpretable surrogate model comprises a polynomial regressor, a shallow decision tree, or a sparse linear model.

9. The genetic circuit system of claim 1, wherein the plurality of layers comprises:
an input layer comprising a first plurality of gene regulatory nodes corresponding to a first subset of genes; and
a first intermediate layer comprising a second plurality of gene regulatory nodes corresponding to a second subset of genes.

10. The genetic circuit system of claim 9, wherein the first subset of genes comprises creb3l1, eid1, ctsf, ndufs5, tgm2, adapt15, and tk1.

11. The genetic circuit system of claim 9, wherein the second subset of genes comprises sdhaf3 and psat1.

12. The genetic circuit system of claim 9, wherein the plurality of layers further comprises a second intermediate layer comprising a third plurality of gene regulatory nodes corresponding to a third subset of genes, and the third subset of genes comprises fam83d.

13. The genetic circuit system of claim 1, wherein the perturbation simulation module is configured to apply the plurality of scaling factors.

14. The genetic circuit system of claim 1, wherein the data processing module is further configured to perform log transformation, normalization, and/or variance-based filtering on the gene expression data prior to input to the feature selection module.

15. An engineered CHO cell, comprising an overexpressed sdhaf3 gene, an overexpressed psat1 gene, and an overexpressed fam83d gene.

16. The engineered CHO cell of claim 15, wherein a Tes gene and/or a Uggt gene thereof is knockout.
